(19) United States
(12) Patent Application Publication
(10) Pub. No.: US 2024/0415752 A1
(43) Pub. Date: Dec. 19, 2024

(54) A personal care composition comprising vitamin K2 and hydroxystearic acid

(71) Applicant: Conopco, Inc., d/b/a UNILEVER, Englewood Cliffs, NJ (US)

(72) Inventors: Permita Bose, Maharashtra (IN); Anita DAMODARAN, Bangalore (IN); Xuelan GU, Shanghai (CN); Annu KUMARI, Keonjhar (IN); Tingyan MI, Shanghai (CN); Nirmala Santosh NAIR, Bangalore (IN); Ashwini SADAWARTE, Mumbai (IN); Satish KUMAR VENKATESH, Karnataka (IN)

(73) Assignee: Conopco, Inc., d/b/a UNILEVER, Englewood Cliffs, NJ (US)

(21) Appl. No.: 18/700,528

(22) PCT Filed: Oct. 10, 2022
(86) PCT No.: PCT/EP2022/078160

§ 371 Date: Apr. 11, 2024

(30) Foreign Application Priority Data
Oct. 13, 2021 (WO) PCT/CN2021/123468
Nov. 29, 2021 (EP) 21210941.7

(51) Int. Cl. A61K 8/365; A61K 8/67; A61Q 19/02

(52) U.S. Cl. CPC A61K 8/365; A61K 8/67; A61Q 19/02; A61K 2800/592

(57) ABSTRACT

The present invention relates to a personal care composition which brightens skin. It more particularly relates to a topical composition which is effective against hyperpigmentation especially in the under-eye region. This is achieved by including in a topical composition. Vitamin K2 and hydroxystearic acid (ISA).

### FIELD OF THE INVENTION

The present invention relates to a personal care composition which brightens skin. It more particularly relates to a topical composition which is effective against hyperpigmentation especially in the under-eye region.

### BACKGROUND OF THE INVENTION

Most people consider skin appearance especially on the face as one of the key indicators of their own beauty and health. Having an even skin tone which is bright and free of blemishes is thus desired by many. The degree and evenness of pigmentation of the skin is affected by factors like age, hormonal changes, occurrence of acne and exposure to sunlight and pollution. These can cause spots or freckles, hyper-pigmentation on certain localised areas of skin and also under-eye dark circles. Many people believe that certain life-style factors like hydration (quantity of water consumed), the type of food and quantity and quality of sleep also has an effect on skin appearance including the dark circles under the eyes. Since people are aware that changes like leading a healthier lifestyle may take a long time for evening out skin appearance, they rely on cosmetic solutions to give them temporary solution to blemishes on their skin. They also seek such products to reduce the skin darkening caused by exposure to sunlight. To meet this need, many attempts have been made to develop products that reduce pigment production in the melanocytes.

The present inventors have been working in this area for a long time and have also filed several patent applications and produced various cosmetic products which are in the market. In the present invention, they were specifically looking for cost effective solution to the problem of under-eye dark circles which could also be used as a general cosmetic product for delivering even and bright skin tone which could be used on any exposed skin surface.

They are aware that various vitamins have been suggested as ingredients to be included in topical compositions for delivering even and bright skin appearance. Various compositions comprising vitamin B3 have been patented by the present applicants for similar benefits. They are aware that Vitamin K has been suggested for such benefit especially for under-eye dark spots.

WO03/101415 (The Boots Co.) discloses a composition comprising, as active ingredient, vitamin K, the vitamin K is present as a complex with a cyclodextrin. The cyclodextrin is most preferably beta-cyclodextrin and the vitamin K is most preferably vitamin K1. The composition may have a generally beneficial effect on the skin, either in a therapeutic or merely cosmetic sense, e.g. in the improving the appearance of the skin or the treatment of bruising and other skin conditions associated with deposition of discolouring material beneath the skin. One particular use of the composition is for improving the appearance of the skin around the eyes, in particular a method for the cosmetic treatment of dark areas (“shadows”) in the vicinity of the eyes.

FR2885803 (L′Oral, 2006) discloses compositions intended for topical application to the skin or the lips comprising, in a physiologically acceptable medium, (a) at least one vitamin K, one of its derivatives or precursors or an extract containing it, (b) a UV filter, and (c) at least one agent chosen from soft focus fillers, fluorescent agents, optical brighteners, goniochromatic pigments, reflecting particles and their mixtures.

The present inventors realised that vitamin K is very expensive and inclusion of it in skin care products to get the desired benefits would make the products very costly and out of reach of most consumers. They thus set about solving the problem of how to use minimum amount of vitamin K and use certain additives such that they would interact synergistically to provide the desired end benefit to the consumer which is reproducible and perceptible over a large portion of the population. To their utter surprise, they found a very selective agent which is a very widely available fatty acid viz. hydroxystearic acid (HSA) which when combined with very minimal amount of vitamin K2, interacted synergistically to boost the efficacy. The present applicant have patented (EP2285342) combinations of 12-HSA with other agent like niacinamide for skin lightening benefits. However, in the present case, many other similar agents known for providing this benefit were tried but surprisingly only HSA was able to interact synergistically with vitamin K2, such that a cost effective solution could be provided where vitamin K2 could be used in very minimal quantity to get the desired benefit.

It is thus an object of the present invention to provide for a solution to the problem of hyperpigmentation and uneven skin appearance.

It is another object of the present invention to provide for such a solution specifically for under-eye dark circles.

It is yet another object of the present invention to provide such solution which is cost effective.

### SUMMARY OF THE INVENTION

The first aspect of the present invention relates to a personal care composition for providing brightness to skin comprising vitamin K2 and hydroxystearic acid (HSA).

Another aspect of the present invention relates to a method of providing brightness to skin preferably to the undereye portion comprising the step of applying a composition of the first aspect on to skin.

### DETAILED DESCRIPTION OF THE INVENTION

These and other aspects, features and advantages will become apparent to those of ordinary skill in the art from a reading of the following detailed description and the appended claims. For the avoidance of doubt, any feature of one aspect of the present invention may be utilised in any other aspect of the invention. The word “comprising” is intended to mean “including” but not necessarily “consisting of” or “composed of.” In other words, the listed steps or options need not be exhaustive. It is noted that the examples given in the description below are intended to clarify the invention and are not intended to limit the invention to those examples per se. Similarly, all percentages are weight/weight percentages unless otherwise indicated. Except in the operating and comparative examples, or where otherwise explicitly indicated, all numbers in this description and claims indicating amounts of material or conditions of reaction, physical properties of materials and/or use are to be understood as modified by the word “about”. Numerical ranges expressed in the format “from x to y” are understood to include x and y. When for a specific feature multiple preferred ranges are described in the format “from x to y”, it is understood that all ranges combining the different endpoints are also contemplated.

The topical composition of the invention is meant to be used for personal care or for cosmetic use and could also be referred to as a personal care composition or a cosmetic composition. By a “personal care composition” as used herein, is meant to include a composition for topical application i.e external surfaces of the skin and/or hair of humans. Such a composition may be classified as leave-on or rinse off, and includes any product applied to a human body for improving appearance, cleansing, odour control or general aesthetics. The composition is preferably of the leave-on type. The composition of the present invention can be in the form of a liquid, lotion, cream, foam, stick, serum, essence or gel. Preferred compositions include leave-on gels, lotions, serum, essence or creams, preferably it is in the lotion or cream form. Most preferred is the cream form.

“Skin” as used herein is meant to include skin on the face and body (e.g., neck, chest, back, arms, underarms, hands, legs and scalp) and especially to the exposed parts thereof. The most preferred part is the under-eye region.

The composition of the invention comprises hydroxystearic acid. It is preferred that the hydroxystearic acid is 10-hydroxystearic acid, 12-hydroxystearic acid or trihydoxystearic acid (e.g. 9,10,13-trihydroxystearic acid) or trihydroxy stearin or compounds that yield one or more molecules of hydroxystearic acid or hydroxystearate on their breakdown like mono, di or tri ester of glycerol with hydroxystearic acid. Of these, 10-hydroxystearic acid, 12-hydroxystearic acid and 9,10,13-trihydroxystearic acid are more preferred, 12-hydroxystearic acid (12-HSA) being most preferred. 12-HSA has the structure as given below:

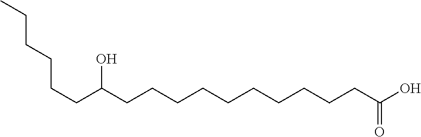 [SMILES: CCCCCCC(O)CCCCCCCCCCC(=O)O]

Hydroxystearic acid is preferably included in 0.01 to 5%, more preferably 0.1 to 3%, further more preferably 0.25 to 2% by weight of the composition.

The composition of the invention comprises Vitamin K2. There are three well known vitamins of the K class. Vitamin K1 is known as phylloquinone, Vitamin K2 is known as menaquinone and Vitamin K3 is known as menadione. The present inventors have found that the synergistic behaviour in combination with HSA is found only with Vitamin K2 and not with the other Vitamins of the K type.

Vitamin K2 has the chemical structure as given below:

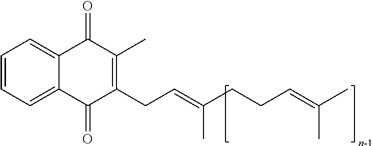

There are ten different types of vitamin K2 existing in nature that are designated as MK-4 to MK-13 depending on its isoprenyl side chain lengths. The present inventors have found that the activity of Vitamin K2 for the purposes of the present invention increases as length of the aliphatic side chain increases. Of these, the present inventors have found that it is preferred that the Vitamin K2 is MK4 to MK7, preferably MK7. It is further preferred that the Vitamin K2 for use in the present invention is encapsulated. Vitamin K2 is preferably included in 0.0001 to 1%, more preferably 0.0001 to 0.1%, by weight of the composition.

The present inventors have found that the Vitamin K2 and HSA are more effective if included in a specific weight ratio. The weight ratio of vitamin K2 to HSA is preferably in the range of 1:50 to 1:500, preferably 1:80 to 1:400, further more preferably in the range of 1:100 to 1:200. The composition preferably includes a skin brightening compound, which preferably is a Vitamin B3 compound, more preferably niacinamide. Niacinamide also known as nicotinamide and as pyridine-3-carboxamide is the active, water soluble form of vitamin B3. When included the composition of the present invention comprises an effective amount of niacinamide, typically in a concentration of 0.001 to 10%, preferably at least 0.01%, more preferably at least 0.1%, still more preferably at least 1% by weight of the composition; Niacinamide is preferably not more than 9%, more preferably not more than 8%, still more preferably not more than 7%, yet more preferably not more than 6%, or even not more than 5% by weight of the composition.

The composition may comprise an organic sunscreen selected from one or both of a UVA sunscreen and a UVB sunscreen. Sunscreens include those materials commonly employed to block ultraviolet light. Illustrative compounds are the derivatives of PABA, cinnamate and salicylate. For example, avobenzophenone (Parsol 1789®) octyl methoxycinnamate and 2-hydroxy-4-methoxy benzophenone (also known as oxybenzone) can be used. Octyl methoxycinnamate and 2-hydroxy-4-methoxy benzophenone are commercially available under the trademarks, Parsol MCX and Benzophenone-3, respectively. The exact amount of sunscreen employed in the compositions can vary depending upon the degree of protection desired from the sun's UV radiation. Additives that reflect or scatter the sun rays may also be employed. These additives include oxides like zinc oxide and titanium dioxide.

The composition of the present invention may further comprise a cosmetically acceptable vehicle, which may act as diluents, dispersants. and/or carriers for the actives used in the composition, so as to facilitate their distribution when the composition is applied to the skin. The cosmetically acceptable vehicle suitable for use in the present invention may be aqueous, anhydrous or an emulsion; aqueous or an emulsion, especially water-in-oil or oil-in-water emulsion being most preferred. Water when present typically makes up the balance of the composition. Preferably water is present in a concentration of 5 to 99%, more preferably from 20 to 80%, still more preferably from 40 and 80% by weight of the composition. The composition of the present invention may be delivered in a cream, lotion or gel form preferably in cream form. A preferred format for the solid form of the composition is a cream, further more preferably one which has a vanishing cream base. Vanishing cream base is one which comprises 3 to 25 wt % fatty acid. The fatty acid for use in preparing the vanishing cream is in addition to hydroxystearic acid which is included as an essential ingredient in the present invention. Optionally, the composition may comprise 0.1 to 10 wt % soap. When included, the fatty acid is preferably a C10 to C22 fatty acid, more preferably a C16 to C18 fatty acid. Most preferably the fatty acids are stearic acid or palmitic acid or a mixture thereof and the soap is preferably the potassium salt of the fatty acid mixture. The fatty acid is often hystric acid which is substantially (generally about 90 to 95%) a mixture of 45% stearic acid and 55% palmitic acid. The most preferred cream is one having 3 to 25 wt % fatty acid and 0.1 to 10 wt % soap.

Preferably, the composition comprises emollients. Examples of emollients that may be used in the leave-on composition include stearyl alcohol, glyceryl monoricinoleate, mink oil, isopropyl isostearate, isobutyl palmitate, isocetyl stearate, oleyl alcohol, isopropyl laurate, hexyl laurate, decyl oleate, octadecan-2-ol, isocetyl alcohol, eicosanyl alcohol, behenyl alcohol, cetyl palmitate, silicone oils such as dimethylpolysiloxane, din-butyl sebacate, isopropyl myristate, isopropyl palmitate, isopropyl stearate, butyl stearate, polyethylene glycol, triethylene glycol, lanolin, cocoa butter, corn oil, cotton seed oil, olive oil, palm kernel oil, rape seed oil, safflower seed oil, evening primrose oil, soybean oil, sunflower seed oil, avocado oil, sesame seed oil, coconut oil, arachis oil, castor oil, acetylated lanolin alcohols, petroleum jelly, mineral oil, butyl myristate, isopropyl linoleate, lauryl lactate, myristyl lactate, decyl oleate, myristyl myristate and mixtures thereof.

Preferably, the composition comprises solvents. Examples of solvents that may be used in the composition include ethyl alcohol, isopropanol, acetone, ethylene glycol mono ethyl ether, diethylene glycol mono butyl ether, diethylene glycol mono ethyl ether and mixtures thereof. The composition may comprise polyhydric alcohols which may be selected from one or more of glycerine, 1,3-butylene glycol, propylene glycol, 1,3-propanediol, pentylene glycol, hexylene glycol, and sorbitol.

Preferably, the composition comprises powders. Examples of powders that may be used in the composition include chalk, talc, fullers earth, kaolin, starch, gums, colloidal silica sodium polyacrylate, tetra alkyl and/or trialkyl aryl ammonium smectites, chemically modified magnesium aluminium silicate, organically modified montmorillonite clay, hydrated aluminium silicate, fumed silica, carboxyvinyl polymer, sodium carboxymethyl cellulose, ethylene glycol monostearate and mixtures thereof.

Preferably, the composition comprises preservatives to protect against the growth of potentially harmful microorganisms. Examples of ingredients that may be used as preservatives in the composition include alkyl esters of para-hydroxybenzoic acid, hydantoin derivatives, propionate salts, and a variety of quaternary ammonium compounds. More preferably, ingredients that may be used as preservative in the composition are sodium benzoate, iodopropynyl butyl carbamate, methylisothiazolinone, iodopropynylbutylcarbamate, phenoxyethanol, methyl paraben, propyl paraben, imidazolidinyl urea, sodium dehydroacetate, ethylhexylglycerin, benzyl alcohol, alkane diols and mixtures thereof. When present in the composition, preservatives are added preferably in an amount 0.001 to 5 wt %, more preferably 0.01 to 3 wt % and most preferably 0.02 to 2 wt %, even most preferably 0.25 to 1.5%.

Preferably, the composition comprises a range of other optional ingredients that include antioxidants, binders, buffering agents, colorants, astringents, fragrance, opacifying agents, conditioners, exfoliating agents, pH adjusters, skin sensates, skin soothing agents, and skin healing agents.

The packaging for the composition of this invention can be a patch, bottle, tube, roll-ball applicator, propellant driven aerosol device, squeeze container or lidded jar.

In a second aspect, the invention relates to use of the composition according to the invention for skin brightening.

In a third aspect, the invention relates to a method of brightening the skin of a human, the method comprising the step of applying the composition according to the invention onto the skin.

The invention will now be illustrated by means of the following non-limiting examples.

### EXAMPLES

### Examples A-D, 1: Melanin Inhibition Rate in Melanocytes

Effect of actives like 12HSA, 4-HR (4-hexyl resorcinol) and Encap MK7 (encapsulated Vitamin K2 of the MK-7 type) were tested individually and in combination. The protocol for the invitro assay is summarized below:

Normal human epidermal melanocytes (NHEM) (Biocell, Xi'an, China) were grown in melanocyte growth medium (MGM) with human melanocyte growth supplement (both from Promocell, Germany). NHEM cells were seeded at a density of 7×10⁴ cells in 2 mL MGM/well in 6-well plates and incubated for 24 hours in an incubator at 37° C. with 5% CO₂. When reaching 50% confluency, cells were irradiated with UVB at 14 mJ/cm² and after UV irradiation, media was replaced with fresh media containing test materials. Every 24 hours in the following 72 hours, the cells were irradiated with UVB again, and the treatment were refreshed.

At the end of the 72 hours incubation period, cell viability was determined using the Alamar Blue method. Briefly, cells were washed twice with phosphate buffered saline (PBS) solution. Freshly prepared 10% Alamar Blue working solution was added into each well including the blank control wells without cells. Plates were incubated for 1 hour at 37° C. in the CO₂ incubator. The Alamar Blue solution was then transferred to a 96-well plate to check fluorescence intensity at Ex/Em 530 nm/590 nm using a fluorescent plate reader.

After cell viability evaluation, cells were further washed with PBS for twice and the cells were detached with 0.25% trypsin and collected with centrifugation. The cell pellet was added with a mixed solution (H₂O:EtOH:Et₂O=2:5:5 v/v) and the mixture was vortexed and left stand still for 30 min. Then the mixture was centrifuged at 1000 r/min for 10 min. The lower fraction was collected and further extracted with a solution containing 10% (v/v) DMSO and 1 M NaOH and incubated at 80° C. in a water bath for 40 minutes. Melanin standard solution was prepared in similarly by adding the same DMSO/NaOH mixed solution to the commercially sourced melanin and incubated at 80° C. for 40 minutes. The extracted melanin and the serially diluted melanin standard solution was then transferred to a 96-well plate and the absorbance at 405 nm was detected using a plate reader. The melanin content was calculated from the melanin standard curve. The melanin concentrations (μg/mL) were then normalized to the relative cell viability. The melanin inhibition rates were calculated using the following equation.

$$Melanin inhibition rate ( \% ) = ( Normalized melanin {content}_{( UV control )} - Normalized melanin {content}_{( Test sample )} / ( Normalized melanin {content}_{( UV control )} \times 100 \%$$

The results in terms of melanin inhibition rate is summarized in table-1 below.

TABLE 1
Melanin inhibition rate

| | | Melanin | |
|---|---|---|---|
| | | inhibition rate | |
| | Active | (% inhibition | |
| Example | (concentration, wt %) | against UV) | SEM |
| A | 12HSA (0.00024%) | 25.3 | 3.20 |
| B | Encap MK7 (0.0024%) | 22.1 | 4.16 |
| 1 | 12HSA (0.00024%) + | 36.3^{a,b} | 4.92 |
| | Encap MK7 (0.0024%) | | |
| C | 4-HR (0.00012%) | 38.5 | 3.55 |
| D | 4-HR (0.00012%) + | 33.0 | 4.92 |
| | Encap MK7 (0.0024%) | | |

^{a,}p < 0.05 compared to 12HSA;
^{b}p < 0.05 compared to Encap MK7

The data in the above table indicates that 12HSA is able to boost efficacy of Vitamin K2 while 4-HR does not do so.

### Example-E.2: Effect of Inclusion of the Actives as Per the Invention as Compared to a Control Sample in a Vanishing Cream Base

Compositions as shown in Table-2 below were prepared. The colour of the skin in 3D living skin equivalent was measured using the following protocol.

MelaKuits®, a reconstructed human pigmented living skin equivalent (pLSE) model supplied by Biocell (Xi'an, China), was used to evaluate the skin lightening effect of the prototypes. Samples were applied topically at 2 mg/cm² on day 4 and left on the model until day 7. At day 7, the L* of each model was measured using the DSM II ColorMeter (Cortex Technology ApS, Denmark).

The L* value and the respective P value (based on Student's t-test) is given in the table below.

TABLE 2

| | Example-E | Example-2 |
|---|---|---|
| Ingredients | (wt %) | (wt %) |
| Glycerine | 1.0 | 1.0 |
| Stearic acid | 17.0 | 17.0 |
| Potasssium | 0.57 | 0.57 |
| hydroxide | | |
| Cetyl alcohol | 0.53 | 0.53 |
| Dimethicone | 0.50 | 0.50 |
| Butyl methoxy | 0.4 | 0.4 |
| dibenzoyl methane | | |
| Octyl methoxy | 0.75 | 0.75 |
| cinnamate | | |
| Isopropyl myristate | 0.75 | 0.75 |
| Niacinamide | 1.25 | 1.25 |
| 12-HSA | 1.00 | 1.00 |
| Vitamin K2 | — | 0.005 |
| Water | To 100 | To 100 |
| L* value | 77.69 ± 0.55 | 80.76 ± 0.80 |

| | |
|---|---|
| P value | 0.048 |

The data in the table-2 above indicates that the composition as per the invention (Example-2) provides for significantly lighter colour as measured in a 3D living cell experiment.

1. A personal care composition comprising: vitamin K2 and hydroxystearic acid (HSA), wherein the hydroxystearic acid is 10-hydroxystearic acid, 12-hydroxystearic acid or 9,10,13-trihydroxystearic acid, and wherein the vitamin K2 is encapsulated.

2. The composition as claimed in claim 1, wherein the vitamin K2 is one or more selected from MK4 to MK7.

3. (canceled)

4. The composition as claimed in claim 1, wherein the hydroxystearic acid is 12-hydroxystearic acid.

5. The composition as claimed in claim 1, wherein the composition comprises 0.0001 to 1 wt % vitamin K2.

6. The composition as claimed in claim 1, wherein the composition comprises 0.01 to 5 wt % HSA.

7. The composition as claimed in claim 1 further comprising a skin brightening agent.

8. The composition as claimed in claim 1, wherein the weight ratio of vitamin K2 to HSA is 1:50 to 1:500.

9. The composition as claimed in claim 1, wherein the composition is in a cream, lotion, or gel form.

10. The composition as claimed in claim 9, wherein the cream comprises 3 to 25 wt % fatty acid and 0.1 to 10 wt % soap.

11. The composition as claimed in claim 1 further comprising a polyhydric alcohol selected from one or more of glycerine, 1,3-butylene glycol, propylene glycol, 1,3-propanediol, pentylene glycol, hexylene glycol, and sorbitol

12. The composition as claimed in claim 1 further comprising an organic sunscreen selected from one or both of a UVA sunscreen and a UVB sunscreen.

13. A method of providing brightness to skin comprising the step of applying a composition as claimed in claim 1 to skin.

14. The composition as claimed in claim 1, wherein the vitamin K2 is MK7.

15. The composition as claimed in claim 7, wherein the skin brightening agent is niacinamide.

16. The composition as claimed in claim 1, wherein the weight ratio of vitamin K2 to HSA is 1:80 to 1:400.

17. The composition as claimed in claim 1, wherein the weight ratio of vitamin K2 to HSA is 1:100 to 1:200.

18. The composition as claimed in claim 1, wherein the composition comprises 0.0001 to 0.1 wt % of vitamin K2.

19. The composition as claimed in claim 1, wherein the composition comprises 0.25 to 2 wt % of HSA.

20. The composition as claimed in claim 12, wherein the sunscreen is selected from octyl methoxycinnamate, butyl methoxy dibenzoyl methane, and a combination thereof.
